(19) United States
(12) Patent Application Publication
(10) Pub. No.: US 2014/0134630 A1
(43) Pub. Date: May 15, 2014

(54) RISK ANALYSIS FOR DISEASE DEVELOPMENT

(71) Applicant: Sai Chavala, Durham, NC (US)

(72) Inventor: Sai Chavala, Durham, NC (US)

(73) Assignee: Serrata, LLC, Durham, NC (US)

(21) Appl. No.: 14/110,025

(22) PCT Filed: Apr. 4, 2012
(86) PCT No.: PCT/US2012/032144

§ 371 Date: Dec. 11, 2013

Related U.S. Application Data
(60) Provisional application No. 61/471,837, filed on Apr. 5, 2011.

(51) Int. Cl. C12Q 1/68; G01N 33/543

(52) U.S. Cl. CPC C12Q 1/686; G01N 33/54306
USPC 435/6.12; 435/7.92

(57) ABSTRACT

In certain embodiments, a novel means for identifying the onset or change in level of severity of given disease states, and/or identifying a patient's risk for experiencing the onset or change in level of severity of given disease states is provided. In some embodiments, a predictive model is provided, which can be used to predict an individual's propensity for developing a given disease or for advancing to a certain stage of a given disease.

### FIELD OF THE INVENTION

The present invention relates to methods for diagnosing and/or monitoring disease progression and to diagnostic kits to facilitate such methods.

### BACKGROUND OF THE INVENTION

Angiogenesis is a process that involves formation and assembly of new blood vessels. In certain situations, angiogenesis is desirable, such as in cardiovascular disease where collateral circulation can prevent damage to the heart muscle. In other situations, angiogenesis is undesirable, such as in age-related macular degeneration (ARMD), where the formation of new blood vessels can lead to decreased vision.

ARMD is the most common cause of central vision blindness in the Western world. There are two types of ARMD: dry (non-neovascular) and wet (neovascular) ARMD. Dry ARMD always precedes wet ARMD. Nine million patients suffer from dry ARMD and approximately 1.4 million patients in the U.S. alone have advanced dry ARMD. Approximately 20% of patients with dry ARMD will develop wet ARMD in their lifetime. The Age-Related Eye Disease Study revealed that an alarming number of patients with high-risk non-neovascular (dry) ARMD will develop vascular (wet) ARMD in five years. Clearly, clinical observations help risk stratify dry ARMD patients, but unfortunately the existing stratification scheme is not precise. The inability to identify the angiogenic switch in a timely manner precludes the development and use of new therapies for delaying and/or preventing the onset of wet ARMD.

Wet ARMD patients often present with abrupt vision loss that can irreversibly impair quality of life. The angiogenic conversion from dry ARMD (development of new blood vessels to result in wet ARMD) is clinically undetected until vision loss ensues. Unfortunately, there are no tests that predict this angiogenic conversion, making it difficult to develop treatments for delay and/or prevention of wet ARMD.

Despite new treatments, wet ARMD causes abrupt irreversible vision loss that can impair quality of life. The ability to prevent or delay the conversion to wet ARMD would have a tremendous impact on preserving vision and maintaining quality of life for ARMD patients. The first step in achieving this goal is to predict the conversion from dry to wet ARMD in a narrow time frame. Identification of dry ARMD patients that are high risk for developing wet ARMD in the near future would allow scientists and clinicians to better test putative agents capable of delaying and/or preventing the conversion from dry to wet ARMD. Some of these agents are already in development but require testing on large groups of high risk dry ARMD patients for long intervals; such patients are not known, as there is no timely way of predicting angiogenic conversion in ARMD. The timely ability to predict the conversion to neovascular ARMD would better enable researchers to develop treatments to delay and/or prevent the angiogenic switch.

Recently, scientists have discovered that precursor cells that participate in angiogenesis as well as endothelial cells can be detected in blood. For example, enumeration of endothelial progenitor cells or circulating endothelial cells has been shown to correlate to severity or disease state in ocular, cardiac, and vascular disease.

Such cells are typically detected by analyzing cell surface markers using standard flow cytometry techniques. However, flow cytometry is a labor intensive method, may be subjective, and is often not reproducible. Further, the cell populations to be analyzed are rare and consist of few cells, and analysis of these cell populations requires sophisticated techniques. Accordingly, an alternative method for analyzing rare cell populations would be desirable, which provides for specific and reproducible results. Further, a means for providing a predictive model for the onset or increase in severity of a given disease state based on these results would be desirable.

### DESCRIPTION OF THE DRAWINGS

FIG. 1 is a graph of assay results for CD34+ and VEGFR-2+ in patients with dry and wet macular degeneration; and

FIG. 2 is a graph of assay results for (CD34+ and VEGFR-2±)/CD34+ in patients with dry and wet macular degeneration.

### SUMMARY OF THE INVENTION

In certain aspects of the invention, a method for identifying the onset or change in level of severity of given disease states, and/or identifying a patient's risk for experiencing the onset or change in level of severity of given disease states is provided.

In some embodiments, the invention provides a method for diagnosing and/or monitoring disease progression in ocular, cardiac, or vascular disease, said method comprising using automated rare cell analysis to analyze a patient sample for the presence of rare cell biomarkers. In certain embodiments, monitoring disease progression comprises: monitoring the extent of a patient's response to treatment, monitoring the time to disease progression, monitoring the progression free time of morbidity, or monitoring the progression free time to mortality or significant loss of vision.

In some embodiments, the invention provides a method for the monitoring the treatment efficacy of an individual having an ocular, cardiac, or vascular disease, said method comprising using automated rare cell analysis to analyze a patient sample for the presence of rare cell biomarkers. In certain embodiments, treatment efficacy is based on duration, clinical efficacy, or side effect profile of treatment response with agents given to improve morbidity or mortality from cardiac disease. In certain embodiments, treatment efficacy is based on duration, clinical efficacy, or side effect profile of treatment response with agents given to reduce vision loss from ocular disease.

In certain embodiments, the method relates to ocular disease, which can be selected, for example, from the group consisting of diabetic retinopathy, diabetic macular edema, sickle cell retinopathy, retinopathy of prematurity, or retinal vein occlusion. In certain embodiments, the method relates to cardiac disease, which can be selected, for example, from the group consisting of ischemic cardiomyopathy, myocardial infarction, ischemic heart disease, acute coronary syndrome, and atherosclerosis. In certain embodiments, the method relates to vascular disease, which can be selected, for example, from the group consisting of cerebrovascular accident (stroke), peripheral vascular disease, and atherosclerotic kidney disease.

The rare cell biomarkers can, in certain embodiments, indicate a change in expression or state that correlates with the risk of progression of ocular disease or with the susceptibility of the disease to a given treatment. In some embodiments, the amount of rare cell biomarker is quantified. The method can, in certain embodiments, further comprise comparing the amount of said biomarker with a reference value.

The composition of the rare cell biomarker can vary. In some embodiments, the rare cell biomarker may comprise a cell expressing one or more specific cell surface antigens. In some embodiments, the rare cell biomarker may comprise a cell surface marker for circulating endothelial cells or circulating endothelial progenitor cells or bone marrow derived cells. In certain embodiments, the rare cell biomarker is the number of VEGFR2⁺CD34⁺CD45⁻ cells. In some embodiments, the biomarker is a percentage of CD34⁺CD45⁻ cells. In further embodiments, the biomarker is the number of G-protein coupled receptor 105 or UDP glucose positive (GPCR-105). In still further embodiments, said biomarker is the number of CD34⁺CD45⁻CD133⁺VEGFR2⁺ or CD34⁺CD45⁻CD133⁺VEGFR2⁻. In other embodiments, the method relates to ocular disease, and the biomarker is the number of CD146⁺CD105⁺CD45⁻.

The patient sample to be analyzed can vary. For example, in some embodiments, the patient sample can be a blood sample or an ocular fluid sample (e.g., aqueous or vitreous fluid).

In another aspect of the invention, a diagnostic kit is provided, comprising at least one means for performing a method as described herein. For example, in certain embodiments, such a kit may comprise a reagent or material selected from antibodies or a reagent or material for monitoring the expression of a biomarker set at the cell surface protein level from patient blood collection.

In a further aspect of the invention, a method for diagnosing or monitoring disease progression in ocular disease is provided, the method comprising analyzing a patient sample for the presence of angiogenic, anti-angiogenic, or both angiogenic and anti-angiogenic cytokine biomarkers, Such cytokine biomarkers can, in some embodiments, be selected from the group consisting of vascular endothelial growth factor, stromal derived factor, erythropoietin, pigment epithelial derived factor, thrombopoietin, and angiomodulin.

### DETAILED DESCRIPTION OF THE INVENTION

In certain embodiments, a novel means for identifying the onset or change in level of severity of given disease states, and/or identifying a patient's risk for experiencing the onset or change in level of severity of given disease states is provided. For example, analysis of certain rare cells in blood samples may be used to identify patients who have developed, who are at risk for developing, or who are at risk for experiencing increased severity of various ocular, cardiac, and/or other vascular diseases. This technique may, in some embodiments, be used in combination with other clinical data to better predict morbidity, and in some cases, even mortality of patients. It may also be used to monitor the efficacy of treatment. In certain cases, this technique may also be useful in the development of therapeutic agents to delay or prevent the onset or change in severity of certain disease states.

In certain embodiments, a predictive model is provided, which can be used to predict an individual's propensity for developing a given disease or for advancing to a certain stage of a given disease. Serum biomarkers provide an attractive adjunct to the current risk stratification scheme that may allow for better precision in predicting, for example, angiogenic conversion. Serum biomarkers are minimally invasive (standard venous blood draw) and can offer significant insight into the timing of the development of angiogenic diseases or disease states.

Although blood/serum can be used according to the invention, other fluids can also be effective in obtaining the desired results described herein. For example, in some embodiments, ocular fluid (namely, aqueous or vitreous fluid) can be analyzed for protein, DNA, and/or RNA levels of anti-angiogenic and/or angiogenic cytokines to serve as biomarkers for macular degeneration. These anti-angiogenic and/or angiogenic cytokines include, but are not limited to, vascular endothelial growth factor, stromal derived factor, erythropoietin, pigment epithelial derived factor, thrombopoietin, and angiomodulin. The analysis can, in certain embodiments, comprise enzyme-linked immunosorbent assay (“ELISA”) and/or polymerase chain reaction (“PCR”) testing on small amounts of fluid to obtain the desired results (e.g., to predict the severity of macular degeneration). It is noted that, depending on the type of patient sample obtained and used, the specific type of analysis can vary. For example, with analysis of an ocular fluid sample, the sample may be analyzed for components other than rare cells (e.g., cytokines), as noted above. Thus, although the discussion and examples provided herein are based primarily on analysis of blood samples for rare cells, analysis of other types of samples, including ocular fluid, for certain other biomarkers are also intended to be encompassed herein and one of skill in the art would be able to modify the preparation and analysis steps accordingly.

In an exemplary embodiment, data is accumulated over a period of time using rare cell analysis to generally equate levels of circulating endothelial cells (CECs) and/or endothelial progenitor cells (EPCs) with disease onset or advancement. Thus, a predictive model may be provided, which sets benchmarks that can be considered to be indicative of the development or advancement of that disease. This predictive model can be used in the clinical setting to quickly and easily monitor patients over time to evaluate disease onset or progression. Such knowledge enables health providers to begin treatments much sooner than is currently possible, which can result in improved results.

There are various means by which CECs and EPCs can be monitored. Although not preferred, flow cytometry, cell culture, and/or related methods may be used. In preferred embodiments, however, automated rare cell analysis is utilized. Automated rare cell analysis is a technique that permits reproducible and specific results when analyzing rare cell populations.

Automated rare cell analysis provides a new method for the detection and/or enumeration of endothelial progenitor cells and/or circulating endothelial cells to evaluate onset or change in severity of a disease state in ocular, cardiac, and vascular disease.

Automated rare cell analysis allows for analysis of blood samples with minimal sample preparation. It allows for the detection of virtually any epitope and provides reproducible and validated results. Further, it can be performed remotely, does not require an experienced operator, and is more objective than other traditional laboratory techniques. Thus, it provides the essential ingredients for widespread clinical implementation. The technique is also approved by the Food and Drug Administration.

An exemplary system for automated rare cell analysis is the Cell Search® system, marketed by Veridex, LLC. This technology, including products and/or associated components thereof, and procedures and instrument systems described herein, are disclosed, for example, by U.S. Pat. Nos. 5,459,073; 5,466,574; 5,512,332; 5,597,531; 5,698,271; 5,849,517; 5,985,153; 5,993,665; 6,120,856; 6,136,182; 6,365,362; 6,551,843; 6,620,627; 6,623,982; 6,645,731; 6,660,159; 6,790,366; 6,861,259; 6,890,426; 7,011,794, 7,282,350 and 7,332,288, which are all incorporated herein by reference. Another exemplary system is the MACSQuant® Analyzer from Milteny Biotec.

According to the invention, the analysis may be based on any rare cell that is indicative of vascularization. For example, in some embodiments, analysis is based on circulating endothelial cells (CECs) and/or endothelial progenitor cells (EPCs).

Circulating endothelial cells are typically described as cells expressing endothelial markers in the absence of hematopoietic and progenitor markers. CECs are usually absent in the blood, but are typically present in the blood of individuals with diseases related to vascularization. For exemplary CEC markers, see, for example, Goon et al., Neoplasia 8(2): 79-88 (2006), which is incorporated herein by reference.

Endothelial progenitor cells belong to a rare cell population that circulates in the peripheral venous blood (and are typically, but not always mobilized form the bone marrow in response to angiogenesis) and that may differentiate to form endothelial cells and/or form new blood vessels. EPCs may be bone marrow-derived cell populations (e.g., myeloid cells, “side population” cells, and mesenchymal cells) or non-bone marrow-derived cells. Identification markers for EPCs include, but are not limited to, CD34+, CD34−, VEGF-2+ (KDR), CD133−, and CD14−. Other exemplary markers for EPCs that may be analyzed according to the present invention are discussed in Urbich et al., Circ. Res. 95:343 (2004); Goon et al., Neoplasia 8(2): 79-88 (2006); and Yoder et al., Blood 109(5): 1801-1809 (2006), and references cited therein, which are all incorporated herein by reference.

In certain embodiments, a predictive model is developed based on clinical data. Generally, clinical data can be obtained by conducting rare cell analysis on a population of patients with and without a given disease/disorder. For example, rare cell analysis can be conducted on a population of individuals with and without a given disease. To develop a predictive model for the onset of a given disease, these results are analyzed to determine if a threshold level of certain rare cells (e.g., CECs or EPCs) exists, above which individuals are considered to have that disease.

Based on this threshold data, the predictive model would enable clinicians to use rare cell analysis to quickly analyze patient samples to determine if the rare cell level is above or below this threshold level. In some embodiments, the rare cell level can be used to develop a risk assessment for a given patient. This risk assessment can be used to develop possible monitoring and/or treatment plans. For example, an individual with somewhat elevated rare cell levels may be monitored more frequently than an individual with very low rare cell levels. In certain embodiments, the predictive model would allow clinicians to monitor patients over time to see if the rare cell level is increasing, which may be indicative of the onset of a vascular disease or the progression of a disease. Thus, according to the present invention, a method is provided wherein rare cell enumeration in combination with clinical data is used to predict the onset or development of given diseases. A good stratification scheme may be the first step in developing preventative treatment, and establishes a platform to intervene with existing or new therapies that can be used to prevent or delay the onset of certain diseases.

Diseases for which this technique is applicable are those diseases that exhibit changes in certain rare cell content at different stages. For example, this technique is particularly applicable to diseases associated with vascularization.

This type of analysis may be a valuable tool for the diagnosis, management, and treatment of ocular diseases. For example, several studies have demonstrated that EPCs correlate to angiogenic phenotype in macular degeneration and diabetive retinopathy. Other ocular diseases that may be relevant to the present invention include, but are not limited to, diabetic macular edema, sickle cell retinopathy, retinopathy of prematurity, and retinal vein occlusion.

In one embodiment, this technique may be used to assess the risk of development of diabetic retinopathy, which is a leading cause of vision loss and blindness. Retinal ischemia from diabetes mellitus leads to the proliferation of new blood vessels, called proliferative diabetic retinopathy. There is currently no predictive model for the transition from non-proliferative diabetic retinopathy to proliferative diabetic retinopathy. Thus, until there is clinical evidence of proliferative diabetic retinopathy, no treatment is typically given. According to the methods provided herein, a predictive model can be developed for the onset of proliferative diabetic retinopathy, enabling clinicians to conduct closer monitoring and/or begin treatment when elevated levels of a particular marker (i.e. above an established benchmark) are detected.

In one embodiment, rare cell analysis of EPCs may be used to determine the likelihood of developing “wet” age-related macular degeneration. Wet age-related macular degeneration is a form of neovascular macular degeneration.

In many cases, patients with “dry” (non-neovascular) macular degeneration have a higher likelihood of developing wet macular degeneration. According to the methods provided herein, a predictive model can be developed for the onset of wet macular degeneration, enabling clinicians to conduct closer monitoring and/or begin treatment when elevated levels of a particular marker (i.e. above an established benchmark) are detected.

Cardiac and vascular diseases can also be monitored based on the methods provided herein. For example, cardiac diseases may include ischemic cardiomyopathy, myocardial infarction, ischemic heart disease, acute coronary syndrome, and atherosclerosis. Vascular diseases may include, for example, cerebrovascular accident (stroke), peripheral vascular disease, and atherosclerotic kidney disease.

### EXPERIMENTAL

The present invention is more fully illustrated by the following example, which is set forth to illustrate the present invention and is not to be construed as limiting thereof.

Patients were recruited, with total enrollment of 26 patients (8 patients having neovascular age-related macular degeneration, and 15 having non-neovascular age-related macular degeneration).

Venous blood was obtained from each patient and the blood was assayed using automated rare cell analysis (using CellSearch® products by Veridex). The results of the assays for CD34+ and VEGFR-2+ are described below.

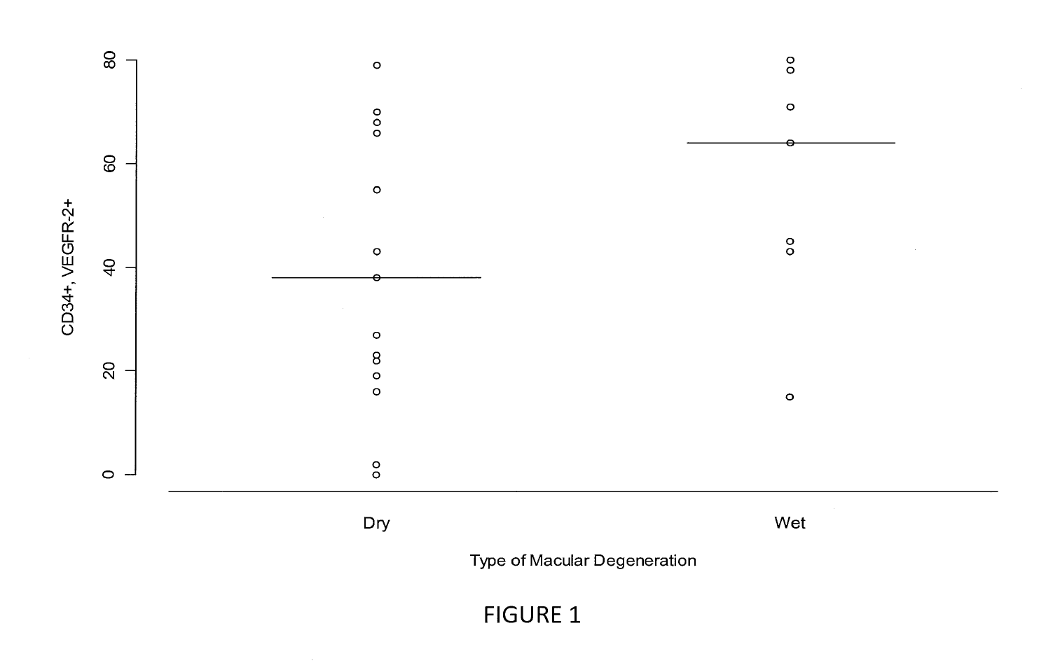

This study shows that patients with wet ARMD generally have a higher percentage of EPCs than patients with dry ARMD. Specifically, FIG. 1 provides results from a CD34+ and VEGFR-2+ double positive assay. The data from the population of dry macular degeneration patients shows a clustering around 20, whereas the data from the population of wet macular degeneration patients shows a higher EPC count, ranging from about 40 upwards.

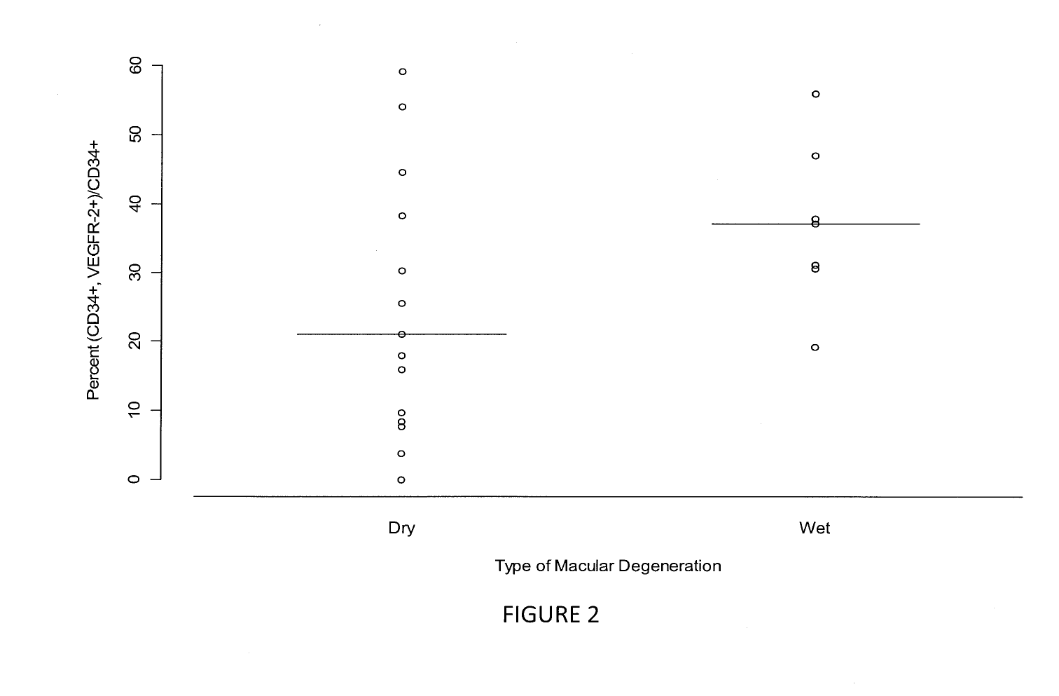

FIG. 2 provides results from a CD34+, VEGFR-2+ assay, giving the percentage of double positive cells among all CD34+ cells. The data from the population of dry macular degeneration patients is clustered around 10, whereas the data from the population of wet macular degeneration patients is clustered around 30-40.

The data suggests that automated rare cell analysis may be a viable method for predicting the likelihood of dry ARMD patients to develop wet ARMD. Patients with dry ARMD will be further monitored over time to develop this technology as an adjunct in developing a predictive model for developing angiogenic eye disease, as well as other ocular, vascular, and cardiac diseases.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A method for diagnosing or monitoring disease progression in ocular, cardiac, or vascular disease or for monitoring the treatment efficacy of an individual having an ocular, cardiac, or vascular disease, said method comprising using automated rare cell analysis to analyze a patient sample for the presence of rare cell biomarkers.

2. (canceled)

3. The method in accordance with claim 1, wherein ocular disease is selected from the group consisting of diabetic retinopathy, diabetic macular edema, sickle cell retinopathy, retinopathy of prematurity, and retinal vein occlusion.

4. The method in accordance with claim 1, wherein cardiac disease is selected from the group consisting of ischemic cardiomyopathy, myocardial infarction, ischemic heart disease, acute coronary syndrome, and atherosclerosis.

5. The method in accordance with claim 1, wherein vascular disease is selected from the group consisting of cerebrovascular accident (stroke), peripheral vascular disease, and atherosclerotic kidney disease.

6. The method in accordance with claim 1, wherein the rare cell biomarkers indicate a change in expression or state that correlates with the risk of progression of ocular disease or with the susceptibility of the disease to a given treatment.

7. The method in accordance with claim 1, wherein monitoring disease progression comprises: monitoring the extent of a patient's response to treatment, monitoring the time to disease progression, monitoring the progression free time of morbidity, or monitoring the progression free time to mortality or significant loss of vision.

8. The method in accordance with claim 1, wherein treatment efficacy is based on duration, clinical efficacy, or side effect profile of treatment response with agents given to improve morbidity or mortality from cardiac disease.

9. The method in accordance with claim 1, wherein treatment efficacy is based on duration of treatment, clinical efficacy of treatment, or side effect profile of treatment response with agents given to reduce vision loss from ocular disease.

10. The method in accordance with claim 1, wherein the amount of said biomarker is quantified.

11. The method in accordance with claim 1, further comprising comparing the amount of said biomarker with a reference value.

12. The method in accordance with claim 1, wherein said biomarker comprises a cell expressing one or more specific cell surface antigens.

13. The method in accordance with claim 1, wherein the patient sample is a blood sample.

14. The method in accordance with claim 1, wherein said biomarker is a cell surface marker for circulating endothelial cells or circulating endothelial progenitor cells or bone marrow derived cells.

15. The method in accordance with claim 1, wherein said biomarker is the number of VEGFR2⁺CD34⁺CD45⁻ cells.

16. The method in accordance with claim 1, wherein said biomarker is a percentage of CD34⁺CD45⁻ cells.

17. The method in accordance with claim 1, wherein said biomarker is the number of G-protein coupled receptor 105 or UDP glucose positive (GPCR-105).

18. The method in accordance with claim 1, wherein said biomarker is the number of CD34⁺CD45⁻CD133⁺VEGFR2⁺ or CD34⁺CD45⁻ CD133⁺VEGFR2⁻.

19. The method in accordance with claim 1 regarding ocular disease, wherein said biomarker is the number of CD146⁺CD105⁺CD45⁻.

20. A method for diagnosing or monitoring disease progression in ocular disease, said method comprising analyzing a patient sample for the presence of angiogenic, anti-angiogenic, or both angiogenic and anti-angiogenic cytokine biomarkers.

21. The method of claim 20, wherein the cytokine biomarkers are selected from the group consisting of vascular endothelial growth factor, stromal derived factor, erythropoietin, pigment epithelial derived factor, thrombopoietin, and angiomodulin.

22. A diagnostic kit comprising at least one means for performing a method according to claim 1.

23. The diagnostic kit in accordance with claim 22, wherein the kit comprises: a reagent or material selected from antibodies, from a reagent or material for monitoring the expression of a biomarker set at the cell surface protein level from patient blood collection.

24. The method in accordance with claim 1, wherein the method is a method for diagnosing or monitoring disease progression in ocular, cardiac, or vascular disease.

25. The method in accordance with claim 1, wherein the method is a method for monitoring the treatment efficacy of an individual having an ocular, cardiac, or vascular disease.
